(19) United States
(12) Patent Application Publication
(10) Pub. No.: US 2010/0298205 A1
(43) Pub. Date: Nov. 25, 2010

(54) METHODS AND COMPOSITIONS FOR THE TREATMENT OF CONDITIONS RELATED TO GASTRIC ACID SECRETION

(76) Inventors: Jie Shen, Irvine, CA (US); Devin F. Welty, Foothill Ranch, CA (US); Diane D. Tang-Liu, Newport Beach, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/639,627

(22) Filed: Dec. 16, 2009

Related U.S. Application Data
(63) Continuation of application No. 10/597,225, filed on Jul. 17, 2006, now abandoned.
(60) Provisional application No. 60/552,501, filed on Mar. 11, 2004.

(51) Int. Cl. A61K 38/06; A61K 31/4439; A61K 31/47; A61P 1/00

(52) U.S. Cl. 514/1.2; 514/338; 514/311

(57) ABSTRACT

A method comprising orally administering to a mammal a proton pump inhibitor, or a pharmaceutically acceptable prodrug thereof, and a compound which modulates the activity of the MRP2 or other transporter proteins involved in efflux of a proton pump inhibitor or a prodrug, is disclosed herein, said method being effective for the prevention or treatment of a disease or condition related to gastric acid secretion. This method applied to compounds which both inhibit and stimulate MRP2 activity or activity of other transporter proteins involved in efflux of a proton pump inhibitor or a prodrug.

Compositions, medicaments, and experimental results related thereto are also disclosed.

### CROSS REFERENCE TO RELATED APPLICATIONS

This is a national stage application under 35 U.S.C. §371 of PCT application PCT/US2005/007015, filed on Mar. 3, 2005, which claims the benefit of Provisional Application No. 60/552,501 filed on Mar. 11, 2004.

### BACKGROUND OF THE INVENTION

### Description of the Related Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042 and 5,708,017. Generally speaking, the benzimidazole-type inhibitors of gastric acid secretion are believed to work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), ESOMEPRAZOLE (U.S. Pat. No. 6,369,085) PANTOPRAZOLE (U.S. Pat. No. 4,758,579), and RABEPRAZOLE (U.S. Pat. No. 5,045,552). Some of the diseases treated by proton pump inhibitors and specifically by the five above-mentioned drugs include peptic ulcer, heartburn, reflux esophagitis, erosive esophagitis, non-ulcer dyspepsia, infection by Helicobacter pylori, alrynitis and asthma.

Whereas the proton pump inhibitor type drugs represent a substantial advance in the field of human and veterinary medicine, they are not totally without shortcomings or disadvantages. For example, it is believed that the short systemic half-life of the drug limits the degree of gastric acid suppression currently achieved. Furthermore, it appears that the short plasma half-life of the drug may contribute to significant gastric pH fluctuations that occur several times a day in patients undergoing PPI therapy. Additionally, PPIs are acid-labile, and in most cases it is necessary to enterically coat the drug in order to prevent the acidic milieu of the stomach from destroying the drug before the drug is absorbed into systemic circulation. Thus, any contribution that might improve the acid stability or plasma half-life of the presently used proton pump inhibitors will be a significant improvement in the art.

As further pertinent background to the present invention, applicants note the concept of prodrugs which is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomsen et al.).

A publication by Sih., et al. (Journal of Medicinal Chemistry, 1991, vol. 34, pp 1049-1062), describes N-acyloxyalkyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N—H group. This publication provides no data nor suggestion regarding the duration of the inhibitory activity of these prodrugs.

U.S. Pat. No. 6,093,734 and PCT Publication WO 00109498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylori effects. PCT Publication WO 02/00166 describes compounds that are said to be nitric oxide (NO) releasing derivatives of proton pump inhibitors of the benzimidazole structure.

Copending U.S. patent application Ser. No. 10/620,252, filed Jul. 15, 2003 discloses prodrugs of the proton pump inhibitor type drugs having an arylsulfonyl group with an acidic functional group attached, which provided improved solubility in physiological fluids and improved cell penetration.

### BRIEF DESCRIPTION OF THE FIGURES

FIG. 1 is a plot of the systemic half-life (T_{1/2}) of proton pump inhibitors omeprazole and lansoprazole, following oral administration of their corresponding prodrugs in dog, as a function of membrane permeability of the prodrugs, measured as the permeability coefficient (Pₐₚₚ) across Caco-2 cells in the apical to basolateral direction.

FIG. 2 depicts compound 1 transport in the basolateral to apical direction across Caco-2 cells in the presence and absence of 50 μM of MK-571 and 250 μM of reduced glutathione.

FIG. 3 depicts the mean omeprazole concentration in blood following oral administration of 16 mg/kg compound 6, with and without co-administration of MK-571 (n=9).

### DETAILED DESCRIPTION OF THE INVENTION

A method comprising orally administering to a mammal a proton pump inhibitor and a compound which modulates the activity of an efflux transporter protein in the gastrointestinal tract epithelium; or prodrugs or pharmaceutically acceptable salts thereof; is disclosed herein; said method being effective for the prevention or treatment of a disease or condition related to gastric acid secretion.

While not intending to limit the scope of the invention in any way, one method comprises orally administering to a mammal a proton pump inhibitor, or a pharmaceutically acceptable salt or prodrug thereof; and a compound which modulates the activity of MRP2, or a pharmaceutically acceptable salt or prodrug thereof.

A composition comprising a proton pump inhibitor and a compound which modulates the activity of an efflux transporter protein in the gastrointestinal tract epithelium; or prodrugs or pharmaceutically acceptable salts thereof; is also disclosed herein.

While not intending to limit the scope of the invention in any way, one composition comprises a proton pump inhibitor, or a pharmaceutically acceptable salt or prodrug thereof; and a modulator of MRP2 activity or a pharmaceutically acceptable salt or prodrug thereof.

The use of a proton pump inhibitor and a compound which modulates the activity of an efflux transporter protein in the gastrointestinal tract epithelium; or prodrugs or pharmaceutically acceptable salts thereof; in the manufacture of a medicament for the prevention or treatment of a condition or disease related to gastric acid secretion afflicting a mammal, is disclosed herein.

While not intending to limit the scope of the invention in any way, one embodiment comprises use of a compound which modulates the activity of MRP2, or a salt or a prodrug thereof; in combination with a proton pump inhibitor, or a salt or a prodrug thereof; in the manufacture of a medicament for the prevention or treatment of a condition or disease related to gastric acid secretion afflicting a mammal, is disclosed herein.

While not intending to limit the scope of the invention in any way, or be bound in any way by theory, it is believed that the methods and composition disclosed herein confer significant advantages to the use and formulation of proton pump inhibitors and their prodrugs for the treatment and prevention of diseases and conditions related to gastric acid secretion.

While not intending to limit the scope of the invention in any way, we have surprisingly discovered that a multidrug resistance-associated protein family member, MRP2, which is expressed at the luminal membrane of the intestinal epithelium, is likely to be one of the transporter proteins responsible for intestinal efflux of proton pump inhibitors and their prodrugs, thus prolonging the systemic half life of these compounds. In other words, it is believed that two-way transport occurs for these compounds, the first transport is absorptive, i.e. from the gut toward the bloodstream, and the second transport is effluxive, i.e. from the gut lining back into the lumen of the gastrointestinal tract. Thus, while not intending to be bound in any way by theory, the effluxive action slows the absorption of the compounds, and the apparent systemic half-live of the proton pump inhibitors is increased. We have surprisingly discovered that compounds believed to modulate the activity efflux transporter proteins of the gastrointestinal tract epithelium, including modulators of MPR2, are capable of altering the net flux of prodrugs of proton pump inhibitors and proton pump inhibitors from the gut into the bloodstream and thus altering the pharmacokinetic profile of proton pump inhibitors. Thus, while not intending to be bound in any way by theory, compounds which modulate the activity efflux transporter proteins in the gastrointestinal tract epithelium are believed to be capable of helping to tune the pharmacokinetics of prodrugs of proton pump inhibitors and proton pump inhibitors themselves, thus improving sustained-release, bioavailability, or peak proton pump inhibitor concentration, according to the particular need. Furthermore, while not intending to be bound in any way by theory, this discovery should enable separate control of the pharmacokinetic and physicochemical properties of these compounds, thus improving flexibility in formulating therapeutic dosage forms.

The term “prodrug” has the meaning previously described herein, and in relation to this disclosure refers to a prodrug of a proton pump inhibitor. The term should be construed broadly, such that if functional groups are present on the prodrug that are capable of forming salts, a salt of such a compound is also considered to be a “prodrug”. The term “proton pump inhibitor” also has the meaning previously described herein.

“A compound a compound which modulates the activity of an efflux transporter protein in the gastrointestinal tract epithelium” is any compound which affects the activity of efflux transporter proteins. This includes any compound which stimulates activity or inhibits activity, regardless of the manner in which this is accomplished. Although the compound may selectively affect the activity of an efflux transporter protein, nonselective compounds may also be used.

“A compound which modulates the activity of MRP2” is any compound, salt, or prodrug which affects the activity of MRP2, whether it stimulates activity or inhibits activity, regardless of the manner in which this is accomplished. Although the compound may selectively affect the activity of MRP2, nonselective compounds may also be used.

In one embodiment, an MRP modulator is used. Known inhibitors of MRP proteins include MK-571, sildenafil (Viagra®), leukotriene C4, gemfibrozil, probenecid, and verapamil. Compounds such as glutathione, which stimulate MRP activity may also be used. Pharmaceutically acceptable salts of these compounds may also be used, and for the purposes herein, the name of any compound applies to both the neutral form and any pharmaceutically acceptable salt.

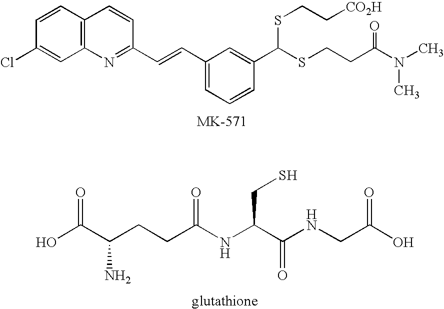

Both proton pump inhibitors and prodrugs may be used in the compositions and methods disclosed herein. While not intending to limit the scope of the invention in any way, commercially available proton pump inhibitors (PPI) include lansoprazole, esomeprazole, omeprazole, pantoprazole, and rabeprazole. Although a prodrug may be prepared from any proton pump inhibitor, it may be desirable to use a prodrug of a commercially available proton pump inhibitor. In situations where the prodrug is derived from one of the commercially available PPIs circumstances related to the individual to which the prodrug is administered are often relevant to the compositions and methods practiced as disclosed herein. For example, if the person to which the prodrug is being administered is known to respond well to omeprazole, then one may consider using a prodrug of omeprazole as disclosed herein. In another situation, a person may have a history of being effectively treated by lansoprazole, in which case one may consider using a prodrug of lansoprazole as disclosed herein. The specific compounds disclosed herein are given merely to provide guidance and direction to one practicing the invention, and are not intended to limit the overall scope of the invention in any way.

In one embodiment the proton pump inhibitor is lansoprazole. In another embodiment the proton pump inhibitor is omeprazole. In another embodiment the proton pump inhibitor is esomeprazole. In another embodiment the proton pump inhibitor is pantoprazole. In another embodiment the proton pump inhibitor is rabeprazole. Other embodiments comprise a prodrug of omeprazole. Other embodiments comprise a prodrug of pantoprazole. Other embodiments comprise a prodrug of rabeprazole. Other embodiments comprise a prodrug of lansoprazole. Other embodiments comprise a prodrug of esomeprazole.

Certain compounds have been shown to be useful as prodrugs in relation to the embodiments disclosed herein. In certain embodiments, the prodrug comprises a sulfonyl moiety. A “sulfonyl” moiety is defined herein as a moiety comprising an SO₂ group, where a sulfur atom is directly covalently bonded to two oxygen atoms. In other embodiments, the prodrug comprises a phenylsulfonyl moiety. The term “phenylsulfonyl” moiety should be broadly interpreted to mean any moiety where the sulfur of the SO₂ group is directly covalently bonded to a carbon that is part of a phenyl ring. The term “phenyl ring” should be broadly understood to mean any ring comprising six carbon atoms having three conjugated double bonds. Thus, a phenylsulfonyl moiety could be monosubstituted, meaning that the sulfonyl group is the only group directly attached to the phenyl ring, or the phenylsulfonyl moiety could have from 1 to 5 additional substituents which are not a hydrogen atom, and are directly attached to a carbon of the phenyl ring. In certain embodiments, the prodrug comprises both a phenylsulfonyl moiety and a carboxylic acid or a pharmaceutically acceptable salt thereof.

Prodrugs may also comprise

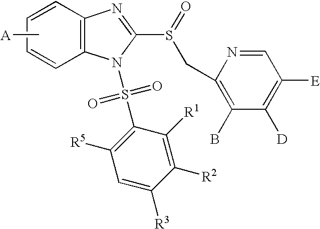

or a pharmaceutically acceptable salt thereof
wherein

### A is H, OCH₃, or OCHF₂;

### B is CH₃ or OCH₃;

### D is OCH₃, OCH₂CF₃, or O(CH₂)₃OCH₃;

### E is H or CH₃;

R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

In another embodiment related to the one just described, R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

In certain embodiments, the prodrug has a structure comprising

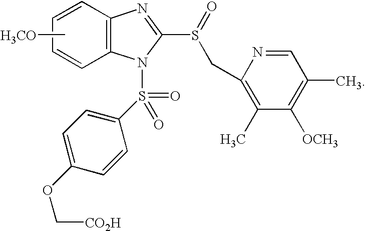

or a pharmaceutically acceptable salt thereof.
Other prodrugs comprise

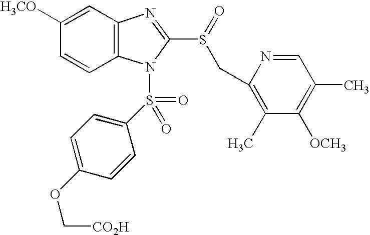

or a pharmaceutically acceptable salt thereof.
Other prodrugs comprise

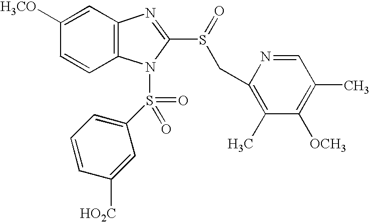

or a pharmaceutically acceptable salt thereof.
Other prodrugs comprise

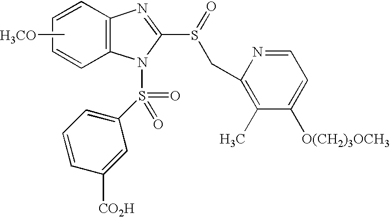

or a pharmaceutically acceptable salt thereof.

In other embodiments, the prodrug has a structure comprising

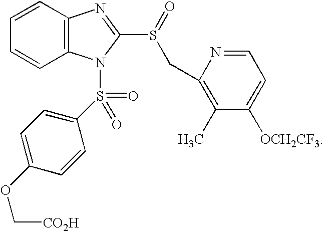

In other embodiments, the prodrug has a structure comprising

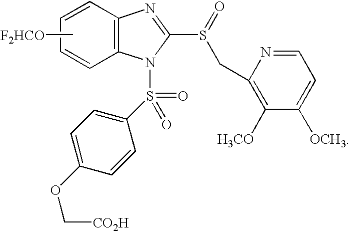

In other embodiments, the prodrug has a structure comprising

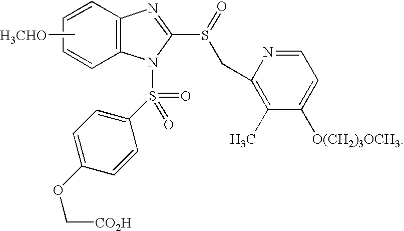

In embodiments comprising a prodrug, the apical to basolateral membrane permeability of the prodrug may vary. The term “apical to basolateral membrane permeability” used in relation to this disclosure refers to the value obtained by carrying out the procedure described in Example 1 herein. In one embodiment the apical to basolateral membrane permeability of the prodrug is less than 1×10⁻⁶ cm/sec. In another embodiment the apical to basolateral membrane permeability of the prodrug is less than 5×10⁻⁸ cm/sec. In another embodiment the apical to basolateral membrane permeability of the prodrug is less than 1×10⁻⁷ cm/sec. In another embodiment the apical to basolateral membrane permeability of the prodrug is less than 5×10⁻⁸ cm/sec.

In certain embodiments, the apical to basolateral membrane permeability of a prodrug as it relates to that of the parent proton pump inhibitor is relevant. In one embodiment the apical to basolateral membrane permeability of the proton pump inhibitor is more than twice the apical to basolateral membrane permeability of the prodrug. In another embodiment, the apical to basolateral membrane permeability of the proton pump inhibitor is more than 10 times the apical to basolateral membrane permeability of the prodrug. In another embodiment the apical to basolateral membrane permeability of the proton pump inhibitor is more than 100 times the apical to basolateral membrane permeability of the prodrug. In another embodiment the apical to basolateral membrane permeability of the proton pump inhibitor is more than 150 times the apical to basolateral membrane permeability of the prodrug.

The prodrugs of the present invention can be prepared by the methods described in the following U.S. Patent documents, all of which are expressly incorporated by reference herein: U.S. Pat. No. 6,093,734; U.S. patent application Ser. No. 09/783,807, filed Feb. 14, 2001; and U.S. patent application Ser. No. 10/620,252, filed Jul. 15, 2003; U.S. patent application Ser. No. 10/487,340, filed Jul. 15, 2003. However, these methods are only given to provide guidance, and are not meant to limit the scope of the invention in any way. One of ordinary skill in the art will recognize that there are many ways in which the prodrugs of the present invention can be prepared without departing from the spirit and scope of the present invention.

Certain embodiments disclosed herein relate to prodrugs comprising an acidic group. An “acidic functional group” as used herein refers to an oxygen containing functional group which has a pKₐ below 10. Thus, while not intending to limit the scope of the claims in any way an acidic functional group may include an organic acid such as a carboxylic acid, a phosphonic acid, or a sulfonic acid.

Acidic functional groups can be in one of two forms, the acid form or the salt form, depending upon whether the particular group has undergone an acid-base reaction. The two forms of these functional groups may also be known by other names. The term “acidic functional group” should be broadly understood to incorporate either the acid or the salt form of the functional group.

A “pharmaceutically acceptable salt” is any salt that retains the activity of the parent compound and does not impart any deleterious or untoward effect on the subject to which it is administered and in the context in which it is administered as compared to the parent compound.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

A disease or condition related to gastric acid secretion is any disease where gastric acid is a cause or a contributing factor, or contributes to a symptom of the diseases, or where inhibition of gastric acid secretion may be helpful in treating or preventing the disease. While not intending to limit the scope of the invention in any way, some examples of such diseases or conditions are peptic ulcer, heartburn, reflux esophagitis, erosive esophagitis, non-ulcer dyspepsia, infection by Helicobacter pylori, alrynitis, and other conditions.

In certain embodiments disclosed herein, the prodrug is not enterically coated. The term “enterically coated” means the prodrug or the dosage form comprising the prodrug is coated by a coating which protects the prodrug from the acids present in the stomach, but which coating disintegrates in the higher pH environment of the intestines. In many dosage forms, small particles of the prodrug are coated with the enteric coating. In other dosage forms, an entire capsule, tablet, or other solid dosage form is coated with the enteric coating. While not intending to be bound in any way by theory, it is believed that the prodrugs disclosed herein are sufficiently stable in the presence of the acidic milieu of the stomach that enteric coating of the prodrug is generally not necessary.

Those skilled in the art will readily understand that for oral administration the compounds of the invention are admixed with pharmaceutically acceptable excipients which per se are well known in the art. Specifically, a drug to be administered systemically, it may be confected as a powder, pill, tablet or the like, or as a syrup or elixir suitable for oral administration. Description of the substances normally used to prepare tablets, powders, pills, syrups and elixirs can be found in several books and treatise well known in the art, for example in Remington's Pharmaceutical Science, Edition 17, Mack Publishing Company, Easton, Pa.

Prodrugs of the present invention can be combined with certain amounts of the proton pump inhibitors to which they are related to provide a drug-prodrug combination, and the combination administered for inhibition of gastric acid secretion. Thus, certain embodiments relate to a mixture of the prodrug and the proton pump inhibitor. Other embodiments relate to the administration of both the prodrug and the proton pump inhibitor. While not intending to limit the scope of these embodiments, it is believed that the proton pump inhibitor (drug) initially inhibits gastric acid secretion of the patient, and as the effective concentration of the proton pump inhibitor (drug) is decreased by metabolism, the prodrug is used to maintain a sustained presence of a therapeutically effective systemic concentration of the proton pump inhibitor. In certain embodiments the ratio of the molar concentration of the prodrug to the molar concentration of the proton pump inhibitor is from 1 to 1000. In other situations, two prodrugs of a proton pump inhibitor are administered to a person to a similar end.

The following examples provide guidance and direction in making and using the invention, and to demonstrate the advantages of the present invention. However, except in the case of Example 1, they are not to be interpreted as limiting the scope of the invention in any way. In the case of Example 1, it should only be interpreted as limiting in relation to those claims where apical to basolateral membrane permeability is used as a limitation.

### Test Compounds

Membrane permeability and oral bioavailability tests were carried out for the compounds shown in Table 1 below. The generic structure, I, is shown as a combination of a proton pump inhibitor (X) and a sulfonyl-bearing moiety which is attached to the proton pump inhibitor to form the prodrug according to the formula below. The identity of each group represented by R¹-R⁵ is shown in the table.

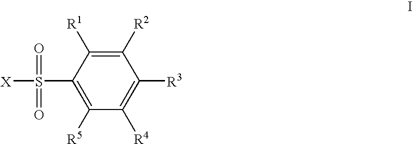

The different possibilities for X are shown below.

TABLE 1

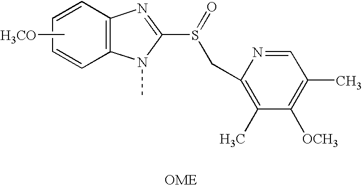

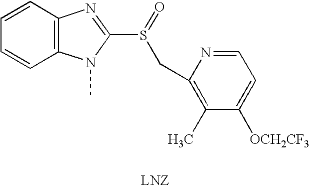

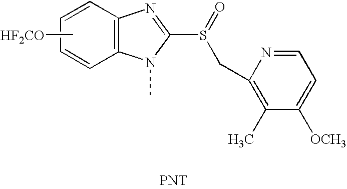

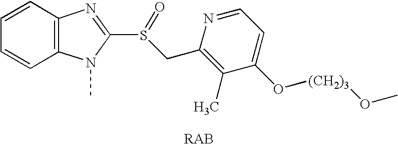

| Compound | X | R¹ | R² | R³ | R⁴ | R⁵ |
|---|---|---|---|---|---|---|
| 1 | OME | H | H | OCH₂CO₂H | H | H |
| 2 | OME | CH₃ | H | OCH₂CO₂H | H | CH₃ |
| 3 | OME | H | H | OCH₂C(CH₃)₂CO₂H | H | H |
| 4 | OME | CH₃ | H | OCH₂C(CH₃)₂CO₂H | H | CH₃ |
| 5 | OME | H | H | CH₂CO₂H | H | H |
| 6 | OME | H | CO₂H | H | H | H |
| 7 | LNZ | H | CO₂H | H | H | H |
| 8 | LNZ | H | CO₂H | OCH₃ | H | H |
| 9 | LNZ | H | H | CH₂CO₂H | H | H |
| 10 | LNZ | H | H | OCH₂CO₂H | H | H |
| 11 | LNZ | H | H | OCH₂C(CH₃)₂CO₂H | H | H |
| 12 | LNZ | H | CH₂CO₂H | CH₂CO₂H | H | H |
| 13 | LNZ | H | CO₂H | H | H | CH₃ |
| 14 | LNZ | H | CO₂H | H | H | OCH₃ |
| 15 | LNZ | CH(CH₃)₂ | H | CH₂CO₂H | H | H |

Compounds were prepared according to procedures described the U.S. patent application Ser. No. 10/620,252, filed Jul. 15, 2003 and U.S. patent application Ser. No. 10/487,340, filed Jul. 15, 2003 incorporated by reference herein.

Omeprazole and lansoprazole were purchased from Sigma (St. Louis, Mo.).

### Example 1

Determination of membrane permeability in all examples described herein was accomplished by the following procedure. This procedure is also used to determine whether a given prodrug falls within the scope of those claims given herein which relate to membrane permeability.

### Materials/Methods

Test System: Cultured Caco-2 cells and MDR1-MDCK cells
Seeding Density: 2×10⁵ cells/cm² in Costar 12 well Transwell™plates
Culture Age: 17-21 days post seeding for Caco-2 cells, 2-3 days post seeding for MDR1-MDCK cells
Source: American Type Culture Collection, Manassas, Va. (Caco-2)
   Dr. Piet Borst at the Netherlands Cancer Institute (Amsterdam, Netherlands) (MDR1-MDCK)
Growth Media Dulbecco's Modified Eagle Media (DMEM) (Gibco BRL) supplemented with 10% fetal bovine serum and 0.1% nonessential amino acids
Dosing Formulation: 10 μM proton pump inhibitor or prodrug in DMEM. Make on the day of dosing.
Assay: LC-MS/MS

### Bi-Directional Transport Experiment:

Caco-2 and MDR1-MDCK cells were seeded on Costar™ 12 mm diameter, 0.4 μm pore size transwell filters, and were cultured at 37° C., 5% CO₂ in a humidified tissue culture chamber.

DMEM was equilibrated as a transport buffer in 37° C. water bath an hour before experiment. The cells were then equilibrated in transport buffer for 1 hr at 37° C.

Dosing solution (10 μM) was prepared by adding a 20 μL aliquot of a 10 mM stock solution of the prodrug to 20 mL of transport buffer.

### Test Conditions:

Transport across Caco-2 or MDR1-MDCK cell monolayer was measured at 37° C., in the apical to basolateral direction (n=3).

Transport buffer was removed from both apical and basolateral compartment of filters. Dosing solution (0.2 mL) was added to the apical compartment of the cell layers on transwell filters, and 0.8 ml fresh pre-warmed transport buffer was added to basolateral compartment. Timing was started for transport, and at 5, 20, and 60 min after transport started, sample fluid (400 μL) was collected from the basolateral compartment. Fresh transport buffer (400 μL) was added back to the basolateral compartment, and the fluid was thoroughly mixed.

Transport samples, dosing solution, and standards (100 μL) each were mixed with 100 μl of a 500 ng/ml internal standard (Lansoprazole-D) for LC-MS/MS analysis, and part of each sample (100 μL) was vortexed and transferred into glass LC-MS/MS vials for analysis.

### Data Analysis

The apparent permeability coefficient (Papp, cm/sec), otherwise known herein as the membrane permeability, is determined from the following relationship:

Papp=J/(ACₒ)

where J (pmol/min) is the transport rate, meaning the rate of prodrug movement through the cell layer, A (cm²) is the filter surface area, and Cₒ (μM) is the initial dosing concentration.

The transport rate J, is calculated as the slope of the linear regression fit for the transport amount over time data using Microsoft Excel® 97 SR-2 (Microsoft Corp. Redmond, Wash.),

### Reference Standard:

Lucifer yellow (LY) was used as a paracellular permeability reference standard to determine integrity of cell layers used in the experiments. LY transport in the apical to basolateral direction was carried out in the same manner as described above. Fluorescence level in basolateral fluid sampled at 5, 20, and 60 min post dose was determined using Fluostar Galaxy (BMG Labtechnologies, Durham, N.C.) at excitation/emission wavelengths of 485/520 nm. A standard curve covering the range from 0.002 to 0.5 mg/mL is constructed to quantify the amount of LY in the transport sample to calculate permeability coefficient (Papp). Papp values below 1×10⁻⁶ cm/sec were considered acceptable and were used to normalize Papp values for test articles across experiments by multiplying the Papp values for the test articles by the factor x according to the following equation, x=(1×10⁻⁶)/(S) where S is the value of Papp obtained for LY.

### Example 2

Oral bioavailability of omeprazole, lansoprazole, pantoprazole, rabeprazole, and test compounds was determined in rats (Sprague-Dawley) and dogs (beagle) by administering an oral solution to the animal and collecting serial blood samples through 24 hr post dose. Blood concentrations of the compounds omeprazole, lansoprazole, pantoprazole, rabeprazole, and test compounds were quantified using an achiral liquid chromatography tandem mass spectrometry method (LC-MS/MS). Systemic pharmacokinetic parameters were determined for omeprazole or lansoprazole using non-compartmental analysis in Watson® version 6.3, available from InnaPhase Corporation, Philadelphia, Pa. Results of the oral pharmacokinetic studies are presented in Tables 2A-2D below.

TABLE 2A
Systemic Omeprazole Half-life in Rats

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.31 |
| 1 | Oral | 10 | 1.7 |
| Omeprazole | Intravenous | 1 | 0.15 |
| 1 | Intravenous | 1 | 0.18 |

Table 2A shows the systemic half-life of omeprazole in rats after oral and intravenous administration of omeprazole and compound 1. Surprisingly, these results show that the systemic half-life of omeprazole after intravenous administration of omeprazole is nearly identical to that after intravenous administration of the prodrug (compound 1). The prodrug was not detected in the bloodstream 5 minutes after it was administered intravenously. These unexpected results demonstrate that in the case of compound 1, systemic conversion of the prodrug to omeprazole does not take an appreciable amount of time compared to the amount of time omeprazole is present systemically. By contrast, slowed absorption of the prodrug from the gastrointestinal tract into the blood unexpectedly prolongs the systemic half-life of omeprazole to a significant extent relative to both the intravenous and oral administration of omeprazole. Table 2B shows a similar effect in dogs. Thus, these results show that oral administration of a prodrug will increase the systemic half-life of a proton pump inhibitor. While not intending to limit the scope of the invention, results that will be discussed later, and which are presented in Table 2D, indicate that a relationship may exist between the membrane permeability of the prodrug and the systemic half-life of the proton pump inhibitor.

TABLE 2B
Systemic Omeprazole Half-life in Dogs

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.70 |
| 1 | Oral | 10 | 2.4 |
| Omeprazole | Intravenous | 1 | 0.60 |
| 1 | Intravenous | 1 | 1.0 |

Table 2C summarizes the systemic half-lives of the prodrugs and the PPIs for compounds I-42 in dogs and rats. While not intending to be limited or bound in any way by theory, these results demonstrate that slow absorption of the prodrug from the gastrointestinal tract can contribute to an increase in the systemic half-life of the proton pump inhibitor. For many of the prodrugs in the table, the systemic half-life of the prodrug (i.e. the intact prodrug molecule) is either very short relative to the systemic half-life of the proton pump inhibitor, or is so short that the intact prodrug cannot be detected in the blood, and thus the half-life cannot be detected (NC). By contrast, however, for many of these same prodrugs, the measured systemic half-life of the proton pump inhibitor is significantly increased relative to the orally administered prodrug. Since the hydrolysis of the prodrugs in the blood does not contribute significantly to the increased systemic half-life of the proton pump inhibitors, it follows that the absorption of the prodrug from the gastrointestinal tract is slowed sufficiently to prolong the systemic half-life of the proton pump inhibitor. Thus, while not intending to be bound or limited in any way by theory, in the case of these particular prodrugs, it is the absorption step rather than the hydrolysis step that is the rate-limiting step of the pharmacokinetic process. In other words, the gastrointestinal tract, rather than the bloodstream, acts as the depot for the prodrug.

TABLE 2C
Systemic Half-Life of Prodrugs and PPIs in Dogs and Rats

| | Dog | | | |
|---|---|---|---|---|
| | T_{1/2} | | Rat | |
| Compound | Prodrug | T_{1/2} PPI | T_{1/2} Prodrug | T_{1/2} PPI |
| Omeprazole | | 0.696 (0.116) | | 0.308 |
| 1 | NC | 2.08 (1.19) | NC | 2.4 |
| 2 | 0.113 | 1.61 | | |
| | (n = 1) | | | |
| 3 | 0.311 | 0.813 | NC | 1.76 (0.93) |
| 4 | 1.26 | 0.837 | 0.342 | 0.708 (0.479) |
| 5 | 0.269 | 1.03 | NC | 1.7 |
| 6 | 0.303 | 1.91 | NC | 1.93 (0.39) |
| 20 | NC | 2.70 (0.62) | | |
| 21 | NC | 0.855 (0.143) | 1.51 (1.44) | 0.523 (0.338) |
| 22 | NC | 3.89 | | |
| 23 | NC | 1.22 | NC | 2.72 (1.35) |
| 24 | | 1.37 | NC | 0.384 |
| 25 | NC | 1.03 | | |
| 26 | 1.19 | 0.881 | | |
| 27 | 0.117 | 1.10 | NC | 2.17 (0.53) |
| | (n = 1) | | | |
| 39 | | | NC | 1.50 (1.18) |
| 40 | | | NC | 2.69 (0.76) |
| 41 | | | NC | 0.761 (0.497) |
| 42 | | | 0.521 | 1.47 (0.29) |
| Lansoprazole | | 0.573 (0.150) | | 0.510 (0.168) |
| 7 | 0.206 | 0.893 | NC | 1.93 (1.41) |
| 8 | NC | 1.08 | NC | 1.80 (1.20) |
| 9 | NC | 0.894 | NC | 0.341 (0.151) |
| 10 | NC | 0.989 (0.307) | | |
| 11 | NC | 0.873 (0.288) | NC | 0.933 (1.009) |
| 12 | NC | 0.931 | | |
| 13 | 0.122 | 1.77 | NC | 2.35 (1.22) |
| 14 | 0.118 | 1.39 | | 0.536 (0.217) |
| 15 | NC | 0.923 | | |
| 16 | NC | 1.00 | NC | 1.86 (0.74) |
| 17 | 1.49 | 1.13 | | |
| 18 | 0.0899 | 0.909 | | |
| 19 | 1.84 | 0.484 | | |
| 34 | | | NC | 1.11 (0.71) |
| 35 | | | NC | 1.84 (0.87) |
| 36 | | | NC | 0.389 (0.085) |
| 37 | | | NC | 2.19 (0.80) |
| 38 | | | 1.04 (0.35) | 1.43 (0.42) |
| Pantoprazole | | 0.743 | | 0.696 (0.116) |
| 28 | NC | 2.61 | NC | 1.45 (0.73) |
| 29 | NC | 0.958 | NC | 1.01 (0.30) |
| Rabeprazole | | 0.369 | | |
| 30 | 1.12 | 0.491 | | |
| 31 | 0.843 | 0.855 | | |
| 32 | 0.526 | 1.52 | | |
| 33 | 0.746 | 0.894 | | |

Values in parenthesis indicate the standard deviation, when obtained.
NC: plasma concentration of prodrug was too low to calculate half-life, or undetected.

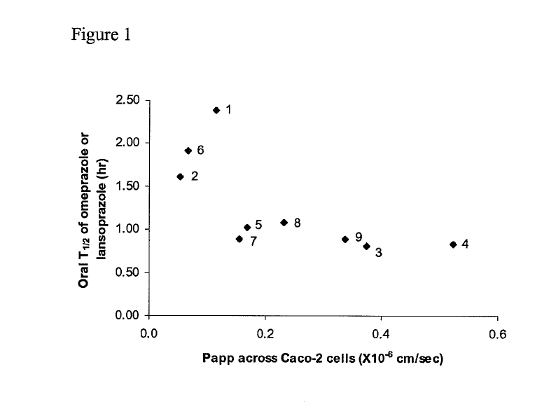

The results in Table 2D demonstrate that apical to basolateral membrane permeability correlates with the systemic half-life of a PPI after oral administration of a PPI or a prodrug. They also demonstrate that apical to basolateral membrane permeability is a good predictive test for how much a given prodrug will increase the systemic half-life of a PPI because the data shows that decreasing the membrane permeability of a prodrug increases the systemic half-life of the PPI. It should be noted that there is some scatter in the data, which is believed to be due to the relatively large random error in determining the systemic half-life. However, FIG. 1 is a plot that graphically demonstrates that despite the scatter, as a general trend, systemic half-life of a PPI resulting from oral administration of its prodrug increases with decreasing membrane permeability of the prodrug.

TABLE 2D
Membrane permeability of proton pump
inhibitors and their prodrugs, and their systemic half-life
in dogs after their oral administration.

| | | Permeability | |
|---|---|---|---|
| Compound | Parent PPI | (×10⁻⁶ cm/sec) | t_{1/2} (hours) |
| Omeprazole | — | 13 | 0.70 |
| 1 | Omeprazole | 0.12 | 2.4 |
| 2 | Omeprazole | 0.054 | 1.6 |
| 3 | Omeprazole | 0.38 | 0.81 |
| 4 | Omeprazole | 0.52 | 0.84 |
| 5 | Omeprazole | 0.17 | 1.0 |
| 6 | Omeprazole | 0.067 | 1.9 |
| Lansoprazole | — | 15 | 0.57 |
| 7 | Lansoprazole | 0.16 | 0.89 |
| 8 | Lansoprazole | 0.23 | 1.1 |
| 9 | Lansoprazole | 0.34 | 0.89 |

### Example 3

To more completely understand the membrane permeability results presented in the previous examples, transport across Caco-2 or MDR1-MDCK cell layers in both the apical to basolateral (A to B) and basolateral to apical (B to A) directions was measured (n=3-4) for compounds 1 and 6 using the methods described in Example 1. Briefly, dosing solution containing the test compounds at 10 μM was applied to either the apical or the basolateral side of the cell layer, while the receiver compartment was bathed in DMEM low glucose medium (free of FBS) as the transport buffer. The cells were incubated at either 37° C. or 4° C. At 5, 20, and 60 min post dose, aliquots were sampled from the receiver compartment. Each time after sampling, same volume of fresh transport buffer was immediately added back to the receiver compartment and mixed well with the remaining fluid.

Pₐₚₚ values in the A to B and B to A directions in Caco-2 and MDR1-MDCK cells for the three different test compounds are listed in Table 3. In Caco-2 cells, all compounds demonstrated preferential transport in the B to A as compared to the A to B direction at 37° C., corresponding to efflux into intestinal lumen in vivo. In other words, while not intending to be bound in any way by theory, these results suggest that following absorption, the cells lining the gastrointestinal tract are able to transport the compounds back into the gastrointestinal tract lumen in the opposite direction of absorption.

When transport was measured for compound 1 in the P-gp over-expressing cell line MDR1-MDCK cells, transport in the efflux direction was not greater. While not intending to be bound in any way by theory, this result suggests P-gp was not involved in the efflux of the prodrugs in Caco-2 cells. Thus, while not intending to be bound in any way by theory, as shown in Example 4, it appears that the activity of MRP2 and other transporter proteins are responsible for the preferred transport of the compounds in the efflux direction, and consequently, contributes to the reduced absorption rate of the prodrugs and the increased systemic half-life of the PPIs observed when prodrugs are administered orally.

TABLE 3
Permeability coefficient estimation for omeprazole, compound 1, and
compound 6 across Caco-2 and MDR1-MDCK cells.

| | Caco-2 cells | | | MDR1-MDCK cells | | |
|---|---|---|---|---|---|---|
| | | | Ratio | | | Ratio |
| | | | Papp | | | Papp |
| | Papp AB | Papp BA | BA/ | Papp AB | Papp BA | BA/ |
| Com- | (cm/sec × | (cm/sec × | Papp | (cm/sec × | (cm/sec × | Papp |
| pound | 10⁻⁵) | 10⁻⁵) | AB | 10⁻⁵) | 10⁻⁵) | AB |
| OME | 1.26 | 1.63 | 1.29 | 2.60 | 1.71 | 1.52 |
| 1 | 0.0165 | 0.0415 | 2.52 | 2.39 | 1.61 | 0.67 |
| 6 | 0.0172 | 0.144 | 8.37 | — | — | — |

### Example 4

Modulating reagents for the multidrug resistant-associated MRP protein were tested in vitro to determine their effect upon transport of compound 1 in Caco-1 cells. In this experiment, dosing solution containing 10 μM of compound 1 was applied to the basolateral compartment of Caco-2 cell layers (n=4) in the presence and absence of MK-571 (50 μM) and glutathione (250 μM) in the apical compartment. MK-571 is a known specific inhibitor for the MRP family [Walgren R A, Karnaky K J Jr, Lindenmayer G E, and Walle T. Efflux of dietary flavonoid quercetin 4′-beta-glucoside across human intestinal Caco-2 cell monolayers by apical multidrug resistance-associated protein-2. J Pharmacol Exp Ther 2000; 294:830-6] whereas reduced glutathione (GSH) has been shown to stimulate MRP transport activity [Van Aubel R A, Koenderink J B, Peters J G, Van Os C H, and Russel F G. Mechanisms and interaction of vinblastine and reduced glutathione transport in membrane vesicles by the rabbit multidrug resistance protein MRP2 expressed in insect cells. Mol Pharmacol 1999; 56:714-9]. The cells were incubated at 37° C. At 5, 20, and 60 minutes postdose, samples were taken from the apical chamber.

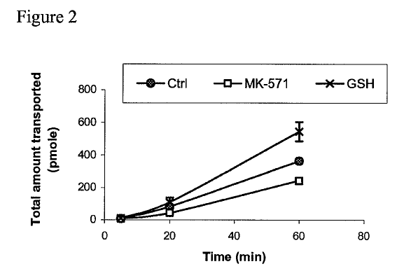

As shown in FIG. 2, in the presence of MK-571 in the apical compartment, transport in the B to A direction was significantly reduced compared to control (Papp=5.21±0.50×10⁻⁶ cm/sec versus control Pₐₚₚ=8.17±0.38×10⁻⁶ cm/sec, p<0.001). On the other hand, presence of glutathione increased B to A transport by over 60% (Papp=13.0±1.7×10⁻⁶ cm/sec, p<0.002). While not intending to be bound in any way by theory, the inhibition and enhancement of compound 1 B to A transport by MK-571 and GSH respectively suggest that compound 1 is a substrate of MRP2 and is transported by MRP2 in the basolateral to apical direction. While not intending to be limit the invention in any way, or be bound by theory, the in vivo implication of this finding is that MRP2 or other transporter proteins expressed at the luminal membrane of intestinal epithelial cells may efflux compound 1 in the export direction. Thus, while not intending to be bound by theory, as compound 1 is continuously absorbed and effluxed in the GI tract, its residence time is effectively prolonged and its absorption time window expanded. Further, while not intending to be bound by theory, or limit the scope of the invention in any way, these results suggest that MRP2 modulators or modulators for other transporter proteins may be used to modify the rate the prodrugs are effluxed, thus modifying the absorption time window, as well as the maximum concentration and the plasma half-life of the PPI.

### Example 5

### In Vivo Pharmacokinetic Study on Compound 6

While not intending to limit the scope of the invention, or be bound in any way by theory, the following in vivo study demonstrates how MRP2 modulators can be used to alter the systemic half life of a PPI after oral administration of a prodrug. Male Sprague-Dawley rats (200-220 g) cannulated in both jugular and femoral veins were purchased from Charles River Laboratories (Wilmington, Mass.). Two treatment groups with nine animals per group were dosed orally with 16 mg/kg compound 6 in a solution. One group of animals were co-administered an oral dose of 10 mg/kg MK-571 in a solution. Blood samples were collected at 5, 10, 20, 40 minutes, 1, 2, 4, 6 and 8 hours post dosing, mixed with five volumes of acetonitrile, and stored frozen at below −70° C. until bioanalysis.

Both in vitro transport and in vivo blood samples were analyzed by LC-MS/MS for concentration of omeprazole and the prodrugs, with detection range of 1-1000 ng/mL. Deuterated omeprazole served as the internal standard.

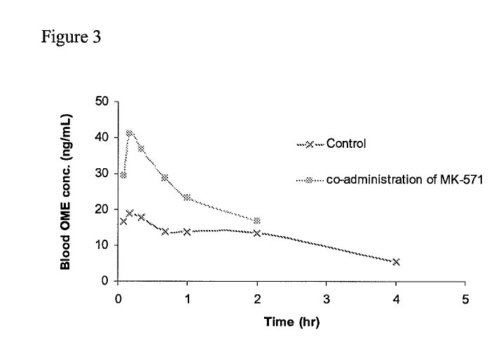

FIG. 3 shows mean blood omeprazole concentration following dosing. With MK-571 co-administration, maximum blood concentration of omeprazole was significantly increased, as would be expected if efflux of its prodrug is inhibited in the GI lumen by an inhibitor of MRP2. Interestingly, systemic half-life of omeprazole was shortened significantly also. The numeric results of this experiment are compiled in Table 5. Thus while not intending to be bound in any way by theory, this result indicates that the efflux of a prodrug may contribute to the prolonged oral half life of a parent proton pump inhibitor following oral administration of the prodrugs by prolonging the GI residence time of the prodrug. While not intending to be bound in any way by theory, or limit the scope of the invention in any way, this result also demonstrates MRP2 modulators or modulators for other transporter proteins involved in efflux can be used to alter the pharmacokinetic profile of a proton pump inhibitor.

TABLE 5
Estimated pharmacokinetic parameters for omeprazole following oral
administration of its prodrug compound 6 with and
without co-administration of MK-571 (n = 9).

| | AUC_{0-∞} | | |
|---|---|---|---|
| | (ng · hr/mL) | Cmax (ng/mL) | t½ (hr) |
| Control | 56.2 ± 17.0 | 21.8 ± 12.4 | 1.64 ± 0.44 |
| +MK-571 | 81.0 ± 39.8 | 43.0 ± 23.9* | 1.17 ± 0.41* |

“*” denotes statistically significant difference (p < 0.05).

While not intending to limit the scope of the invention in any way, the results presented herein suggest that there are a number of ways that MRP2 modulators or modulators for other transporter proteins could be used to modify the pharmacokinetics to improve the properties, depending upon the circumstances. For example, adding a compound which stimulates MRP2 activity or activity of other transporter proteins involved in efflux could be used to increase the plasma half life of a proton pump inhibitor by oral administration with a prodrug. Thus, if a particular prodrug has desirable physicochemical properties, but slower absorption is desired, a compound such as glutathione could be administered either separately, or in a single composition with the prodrug. A compound which stimulates MRP2 activity or the activity of other transporter proteins involved in efflux may also be used to improve sustained release of a proton pump inhibitor when it is administered orally, and not as the prodrug. Alternatively, a compound which inhibits MRP2 activity or activity of other transporter proteins involved in efflux may be used to provide rapid onset of action and increase the bioavailability of the PPI when used in conjunction with a prodrug that is absorbed more slowly than is desired. This may be useful in providing a faster acting dosage form.

While not intending to limit the scope of the invention in any way, a non-obvious use of a compound such as MK-571, which inhibits MRP2 activity, would be to enhance sustained release of a proton pump inhibitor by oral administration of a prodrug. For example, FIG. 3 shows that the concentration of the PPI in the blood is higher from about 0-2 hours when MK-571 is administered with the compound 6 as compared to when compound 6 is administered alone. While not intending to be bound by theory, it is believed that the reduced efflux allows greater systemic absorption of the prodrug, thus producing a higher plasma concentration of the PPI. However, the inhibition of the efflux activity need not occur when the prodrug is administered. For example, in the case of compound 6, if MK-571 is administered about 2 hours after the prodrug is administered, one may expect that the absorption would increase, thus increasing the amount of PPI in the blood for as long as the prodrug is available for absorption. Thus, the total bioavailability of the prodrug might be increased, and the uneven control of stomach pH which is associated with PPI use might be improved. Alternatively, a dosage form might be formulated which allows delayed release of the MRP inhibitor. Thus, both the inhibitors and stimulators of MRP2 or other transporter proteins involved in efflux are useful in improving the pharmacokinetic properties of PPIs and their prodrugs through a large variety of compositions and methods.

### Example 6

The physicochemical properties of compound 1 were analyzed. Compound 1 was found to be hygroscopic, in that 9% weight gain was observed for the compound after 14 days of storage at 25° C. at 75% relative humidity.

TABLE 3A
Solubility Profile of Compound
1at 25° C. in Buffered Aqueous Solutions

| | | Solubility |
|---|---|---|
| pH | Buffer Composition | (mg/mL) |
| 1 | 0.1 m HCl | 1.8 |
| 3 | Citric Acid (0.1 M)/ | 0.4 |
| | Na₂HPO₄ (0.2 M) | |
| 5 | Citric Acid (0.1 M)/ | >50 |
| | Na₂HPO₄ (0.2 M) | |
| 7 | sodium phosphate (0.1-0.2 M) | >50 |
| 9 | sodium phosphate (0.1-0.2 M) | >50 |

The solubility profile of compound 1 in at various pH values is presented in Table 3A. This data shows that the aqueous solubility of the compound is significantly enhanced at around pH 5. While not intending to be bound in any way by theory, it is believed that this improvement in solubility is due to the deprotonation of a sufficient quantity of the acid. While not intending to be bound in any way by theory, this suggests that the prodrug should be significantly easier to formulate, particularly in the case of liquid dosage forms, when the pH is around 5 or higher.

TABLE 3A
Stability Profile of Compound 1 at 25° C. in Buffered
Aqueous Solutions

| | | Half-life | | Degradation |
|---|---|---|---|---|
| | Buffer | (t_{1/2}) | Shelf life | Rate Constant |
| pH | Composition | hours | (t_{90%}) hours | (k) 1/hours |
| 1 | 0.1 M HCl | 3.6 | 0.5 | 0.194 |
| 3 | Citric Acid (0.1 | 78.0 | 11.9 | 0.009 |
| | M)/Na₂HPO₄ (0.2 | | | |
| | M) | | | |
| 5 | Citric Acid (0.1 M)/ | 89.2 | 13.6 | 0.008 |
| | Na₂HPO₄ (0.2 M) | | | |
| 7 | sodium phosphate | 286.8 | 43.6 | 0.002 |
| | (0.1-0.2 M) | | | |
| 7.4 | sodium phosphate | 291.2 | 44.3 | 0.002 |
| | (0.1-0.2 M) | | | |
| 9 | sodium phosphate | 23.0 | 3.5 | 0.030 |
| | (0.1-0.2 M) | | | |
| 10 | sodium phosphate | 2.3 | 0.4 | 0.298 |
| | (0.1-0.2 M) | | | |

The aqueous stability data of compound 1 is presented in Table 3B. These results show that, the half-life (t_{1/2}), the shelf-life (t₉₀%), and the rate constant for degradation (k) for compound 1 are significantly improved in the pH range of 3-9. While not intending to be bound in any way by theory, these results suggest that formulation of dosage forms in the pH range of from 3 to 9 should greatly improve the stability of the prodrugs, thus improving shelf-life and facilitating formulation. Further, these results suggest that dosage forms having a pH from 6 to 8 will be particularly useful in certain situations.

Additionally, these results demonstrate that the prodrugs are significantly more stable in acidic and neutral aqueous solutions than the proton pump inhibitors. The stability of omeprazole and other proton pump inhibitors have been reported (Kromer et al., “Differences in pH-Dependent Activation Rates of Substituted Benzimidazoles and Biological in vitro Correlates”, Pharmacology 1998; 56:57-70; and Ekpe et al, “Effect of Various Salts on the Stability of Lansoprazole, Omeprazole, and Pantoprazole as Determined by High Performance Liquid Chromatograpy”, Drug Development and Industrial Pharmacy, 25(9), 1057-1065 (1999)), and while the stability is somewhat buffer dependent, typical half-lives for omeprazole are about 1 hour at pH 5 and about 40 hours at pH 7, which is about 1-2 orders of magnitude shorter than the prodrug half-lives presented in Table 3A. This instability of the proton pump inhibitors has generally necessitated their formulation in enterically-coated dosage forms. Thus, while not intending to limit the scope of the invention in any way, or to be bound in any way by theory, these results suggest that the prodrugs disclosed herein have sufficient stability to allow the gastrointestinal tract to act as a depot for the prodrug, and also have sufficient stability that the use of enteric coatings is not necessary for effective formulation of a dosage form.

### Example 7

A capsule containing 40 mg of compound 6 is administered to a patient suffering from heartburn. Two hours later, a capsule containing 40 mg of MK-571 is administered to the same patient. This therapy is repeated daily, and relief from heartburn is experienced for as long as the therapy continues.

### Example 8

Compound 1 (60 mg) and MK-571 (40 mg) are dissolved by stirring into 5 mL of water at 50° C. The solution is allowed to cool to room temperature, and the entire volume of solution is administered to a patient suffering from heartburn. This procedure is repeated daily, and relief from heartburn is experienced for as long as the therapy continues.

### Example 9

A capsule containing compound 6 (60 mg) and glutathione (200 mg) is administered to a patient having an ulcer. This therapy is repeated daily, and relief from symptoms is experienced for as long as the therapy continues.

What is claimed is:

1. A method comprising orally administering to a mammal
a) a proton pump inhibitor, or a pharmaceutically acceptable prodrug thereof, and
b) a compound which modulates the activity of an efflux transporter protein in the gastrointestinal tract epithelium, or a pharmaceutically acceptable salt or a prodrug thereof,
said method being effective for the prevention or treatment of a disease or condition related to gastric acid secretion.

2. The method of claim 1 wherein said compound inhibits an efflux transporter protein.

3. The method of claim 1 wherein said compound stimulates an efflux transporter protein.

4. The method of claim 1 wherein said efflux transport protein is MRP2.

5. The method of claim 1 comprising a prodrug of a proton pump inhibitor or a pharmaceutically acceptable salt thereof.

6. The method of claim 4 wherein said compound inhibits MRP2 activity.

7. The method of claim 4 wherein said compound stimulates MRP2 activity.

8. The method of claim 6 wherein said compound is MK-571.

9. The method of claim 7 wherein said compound is glutathione.

10. The method of claim 1 comprising 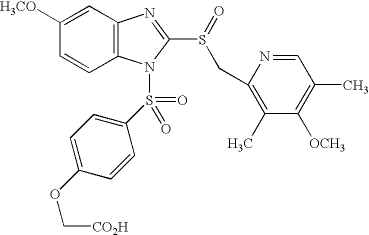
or a pharmaceutically acceptable salt thereof.

11. The method of claim 1 comprising 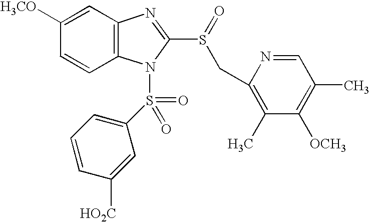
or a pharmaceutically acceptable salt thereof.

12. The method of claim 10 comprising MK-571.

13. The method of claim 10 comprising glutathione.

14. The method of claim 11 comprising MK-571.

15. The method of claim 11 comprising glutathione.

16. A composition comprising a proton pump inhibitor or a prodrug or a pharmaceutically acceptable salt thereof; and a compound which modulates the activity of an efflux transporter protein in the gastrointestinal tract epithelium; or a pharmaceutically acceptable salt or a prodrug thereof.

17. The composition of claim 16 wherein said efflux transporter protein is MRP2.

18. The composition of claim 16 comprising a prodrug of a proton pump inhibitor or a pharmaceutically acceptable salt thereof.

19. The composition of claim 16 comprising 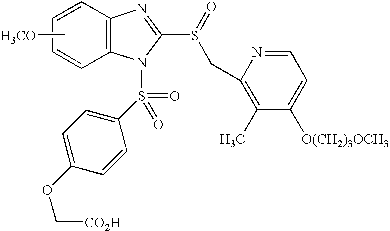
or a pharmaceutically acceptable salt thereof.

20. The composition of claim 16 comprising 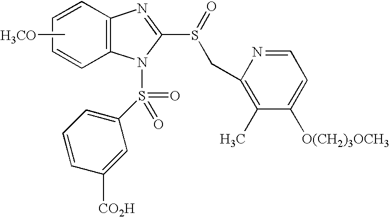
or a pharmaceutically acceptable salt thereof.

21. The composition of claim 19 comprising MK-571.

22. The composition of claim 19 comprising glutathione.

23. The composition of claim 20 comprising MK-571.

24. The composition of claim 20 comprising glutathione.
